(19) United States
(12) Patent Application Publication
(10) Pub. No.: US 2026/0204426 A1
(43) Pub. Date: Jul. 16, 2026

(54) ANALYSIS DEVICE, ANALYSIS METHOD, AND ANALYSIS PROGRAM

(71) Applicant: Hitachi, Ltd., Chiyoda-ku, Tokyo (JP)

(72) Inventors: Yasuaki NAKAMURA, Tokyo (JP); Wataru TAKEUCHI, Tokyo (JP)

(21) Appl. No.: 18/872,487

(22) PCT Filed: Feb. 8, 2023
(86) PCT No.: PCT/JP2023/004084

§ 371 Date: Dec. 6, 2024

(30) Foreign Application Priority Data
Jun. 7, 2022 (JP) 2022-092187

(51) Int. Cl. G16H 50/70; G16H 10/60

(52) U.S. Cl. CPC G16H 50/70; G16H 10/60

(57) ABSTRACT

An analysis device includes: a processor configured to execute a program; and a storage device configured to store the program. The storage device stores a weight for each predictive factor group in a factor group. The analysis device includes an acquisition unit configured to acquire a plurality of pieces of patient data including a value for each factor of the factor group for each patient, and a search unit configured to repeatedly execute selection processing of selecting the factor and the weight, division processing of dividing the plurality of pieces of patient data that are a division target based on the factor and the weight that are selected by the selection processing, and setting processing of setting a patient data group obtained by the division processing as a new division target, thereby executing search processing of searching for a branch condition for dividing the division target by the division processing.

### CROSS-REFERENCE TO RELATED APPLICATION

The present application claims the priority of Japanese Patent Application No. 2022-92187, filed on Jun. 7, 2022, the entire contents of which are incorporated herein by reference.

### TECHNICAL FIELD

The present invention relates to an analysis device, an analysis method, and an analysis program for analyzing data.

### BACKGROUND ART

Conventional medical practice has promoted standardization and guideline creation based on randomized controlled trials, and on the other hand, it has become evident that a treatment is not effective for all patients and there is individual variability. Therefore, current medical practice focuses on pursuit of an optimal treatment selection tailored to an individual characteristic of a patient. For example, a comprehensive medical data analysis system has been disclosed in which patients are classified into subtypes (stratification) based on a patient characteristic and the like, and treatments and outcomes for similar patients are analyzed (see the following PTL 1).

The comprehensive medical data analysis system includes a medical main server including an intelligent medical engine, the intelligent medical engine is communicably coupled to a central database that is a confidential electronic medical record database, and is further communicably coupled to a hospital, a clinic, and other medical resources via a network. The intelligent medical engine receives a large number of medical records from potentially different countries, regions, and continents. The electronic medical records are provided from a hospital, a clinic, and other medical resources, and are supplied into the intelligent medical engine such that medical records of patients can be correlated by global large-scale analysis. The analysis is started by grouping (classifying) the medical records into subgroups of a plurality of levels according to a patient clinical parameter, a disease template, a treatment, and an outcome. When a new patient is input to the system, a parameter and a disease template of the patient are matched with a most similar subgroup for a possibly favorable outcome.

### CITATION LIST

### Patent Literature

PTL 1: WO2015/082555

### Non Patent Literature

NPL 1: Athey, Susan, et al, “Recursive partitioning for heterogeneous causal effects” Proceedings of the National Academy of Sciences 113.27 (2016): 7353-7360.

### SUMMARY OF INVENTION

### Technical Problem

However, in the comprehensive medical data analysis system of PTL 1, the subgroups are not divided based on the treatment effect. In addition, in NPL 1, a factor (predictive factor) related to the treatment and a factor (prognostic factor) not related to the treatment are similarly handled in the estimation of the treatment effect.

An object of the invention is to improve estimation accuracy of a treatment effect.

### Solution to Problem

An analysis device according to an aspect of the invention disclosed in the present application is an analysis device includes a processor configured to execute a program, and a storage device configured to store the program. The storage device stores a weight for each predictive factor group in a factor group. The analysis device includes an acquisition unit configured to acquire a plurality of pieces of patient data including a value for each factor of the factor group for each patient, and a search unit configured to repeatedly execute selection processing of selecting the factor and the weight, division processing of dividing the plurality of pieces of patient data that are a division target based on the factor and the weight that are selected by the selection processing, and setting processing of setting a patient data group obtained by the division processing as a new division target, thereby executing processing of searching for a branch condition for dividing the division target by the division processing.

### Advantageous Effects of Invention

According to a representative embodiment of the invention, estimation accuracy of a treatment effect can be improved. Problems, configurations, and effects other than those described above will be clarified by descriptions of the following embodiments.

### BRIEF DESCRIPTION OF DRAWINGS

FIG. 1 is a diagram showing an example of outcomes of a prognostic factor and a predictive factor.

FIG. 2 is a diagram showing an example in which a population of patients is divided by the predictive factor in a patient characteristic considered to be significantly effective on a treatment effect 1 and weighting the predictive factor during learning.

FIG. 3 is a block diagram showing a hardware structure example of an analysis device.

FIG. 4 is a block diagram showing a functional configuration example of the analysis device.

FIG. 5 is a diagram showing an example of a weight table shown in FIG. 4.

FIG. 6 is a diagram showing an example of a health care DB shown in FIG. 4.

FIG. 7 is a diagram showing an example of a patient data table.

FIG. 8 is a diagram showing an example of an input screen of the analysis device.

FIG. 9 is a flowchart showing an analysis processing procedure example performed by the analysis device.

FIG. 10 is a diagram showing an example of a stratification result.

FIG. 11 is a diagram showing another example of the stratification result.

FIG. 12 is a flowchart showing a detailed processing procedure example of stratification processing (step S902) shown in FIG. 9.

FIG. 13 is a flowchart showing a detailed processing procedure example of branch condition search processing (step S1002) shown in FIG. 10.

FIG. 14 is a box plot diagram showing a prediction error improvement rate as compared with a case before division between a conventional method and Embodiment 1.

FIG. 15 is a flowchart showing a generation processing procedure example of a weight table generated by a generation unit according to Embodiment 2.

FIG. 16 is a histogram showing a search result from a medical literature database.

FIG. 17 is a flowchart showing a generation processing procedure example of a weight table according to Embodiment 3.

### DESCRIPTION OF EMBODIMENTS

### <Outcomes of Prognostic Factor and Predictive Factor>

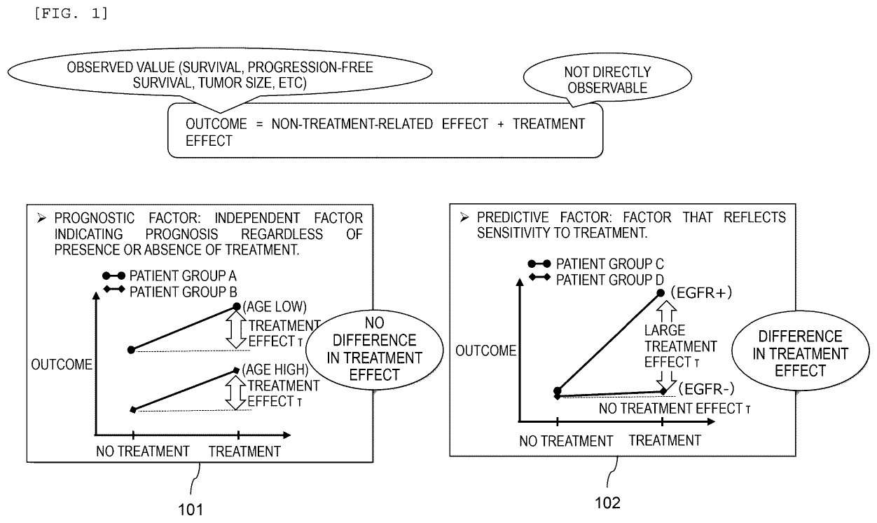

FIG. 1 is a diagram showing an example of outcomes of a prognostic factor and a predictive factor. The outcome is, for example, an observed value such as survival, progression-free survival, or a tumor size, and is a value inherently including a non-treatment-related effect and a treatment effect. The non-treatment-related effect and the treatment effect are not directly observable.

A graph 101 indicates the outcome before and after a treatment of patient groups A and B obtained by grouping a population of patients according to presence or absence of the prognostic factor. A graph 102 indicates the outcome before and after the treatment of patient groups C and D obtained by grouping the population of patients according to presence or absence of the predictive factor.

Each of the prognostic factor and the predictive factor is any factor in a factor group constituting a characteristic of a patient (hereinafter, referred to as a patient characteristic), and is a quantitative variable, that is, a covariate that varies with the outcome. The prognostic factor is an independent factor indicating prognosis regardless of presence or absence of the treatment, and is, for example, an age of the patient. The predictive factor is a factor that reflects sensitivity to the treatment, such as an epidermal growth factor receptor (EGFR), which is a factor showing different treatment effects depending on presence or absence of the predictive factor.

In the graph 101, the patient group A is a set (age low) of patients each having a low value of the prognostic factor indicating the age, and the patient group B is a set (age high) of patients each having a higher value of the prognostic factor indicating the age than that of the patient group A. In the graph 101, although the outcome before and after the treatment varies due to a difference between the patient groups A and B, there is no difference in a treatment effect τ (a difference in the outcome before and after the treatment) between the patient groups A and B.

In the graph 102, the patient group C is a set (EGFR+) of patients each having a large value of the predictive factor indicating EGFR, and the patient group D is a set (EGFR−) of patients each having a smaller predictive factor indicating EGFR than the patient group C. In the graph 102, the outcome before and after the treatment varies due to a difference between the patient groups C and D, and there is also a difference in the treatment effect τ (a difference in the outcome before and after the treatment) between the patient groups C and D. In the graph 102, the treatment effect τ of the patient group C is larger than the treatment effect τ of the patient group D.

Accordingly, by stratifying the population of the patients with the predictive factor such as EGFR, it is possible to support a treatment selection through the state classification for each treatment effect t, but when the population of the patients is not stratified with the predictive factor, the prediction accuracy of the treatment effect τ decreases. Therefore, in the embodiments described below, the prediction accuracy of the treatment effect τ is improved by specifying in advance the predictive factor in the patient characteristic considered to be significantly effective on the treatment effect τ and weighting the predictive factor during learning.

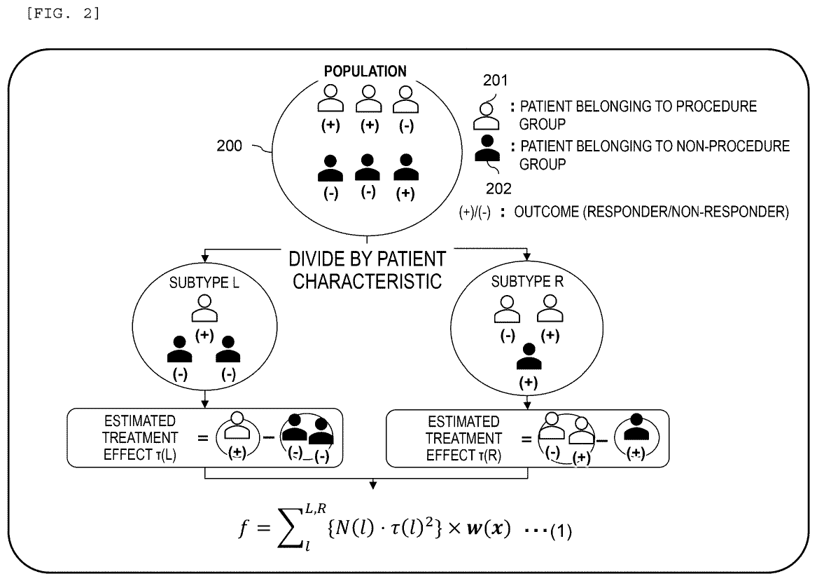

FIG. 2 is a diagram showing an example in which the population of the patients is divided by the predictive factor in the patient characteristic considered to be significantly effective on the treatment effect τ and weighting the predictive factor during learning. A population 200 includes a patient 201 belonging to a procedure group and a patient 202 belonging to a non-procedure group. The procedure group is a set of patients who receive a medical procedure for injury or illness, and the non-procedure group is a set of patients who receive no medical procedure for injury or illness. In addition, (+) indicates a responder and (−) indicates a non-responder. Hereinafter, the patients 201 and 202 who are responders are referred to as patients 201(+) and 202(+), and the patients 201 and 202 who are non-responders are referred to as patients 201(−) and 202(−).

That is, the patient 201(+) is a patient 201 whose injury or illness is cured by a procedure, and the patient 201(−) is a patient 201 whose injury or illness is not cured even when receiving the procedure. In addition, the patient 202(+) is a patient 202 whose injury or illness is cured even when receiving no procedure, and the patient 202(−) is a patient 202 whose injury or illness is not cured without a procedure. In FIG. 2, for simplicity of description, a set of the six patients 201 and 202 is referred to as the population 200.

Here, an analysis device divides the population 200 of the patients into two groups based on a predictive factor x in the patient characteristic considered to be significantly effective on the treatment effect t. One of the groups is referred to as a subtype L, and the other group is referred to as a subtype R.

An estimated treatment effect τ(L) of the subtype L is a difference between an outcome of the patient 201(+) in the subtype L and an outcome of the patients 202(−) in the subtype L, and corresponds to the difference in the treatment effect τ between the patient groups C and D in FIG. 1.

An estimated treatment effect τ(R) of the subtype R is a difference between an outcome of the patients 201(+) and 201(−) in the subtype R and an outcome of the patient 202(+) in the subtype R, and corresponds to the difference in the treatment effect τ between the patient groups C and D in FIG. 1.

By weighting the weight w(x) related to the predictive factor x, which is obtained by dividing the population 200 into the subtypes L and R, to a sum of squares of the estimated treatment effects τ(L) and τ(R), the analysis device learns a loss function f using the following formula (1), or predicts the treatment effect τ of the patient to be predicted based on the loss function f.

$$[ Math 1 ]$$

$$f = {\sum }_{l}^{L , R} \{ N ( l ) \cdot {τ ( l )}^{2} \} \times w ( x ) \qquad ( 1 )$$

l is an index indicating whether a treatment effect τ(l) is of the subtype L or R. In addition, N(l) is the number of samples of the subtype L. Hereinafter, the analysis device shown in FIGS. 1 and 2 will be described in detail as Embodiments 1 to 3.

### Embodiment 1

In Embodiment 1, an analysis device in which a weight w(x) is specified in advance will be described. The invention is not limited to the following embodiments.

### <Hardware Configuration Example of Analysis Device>

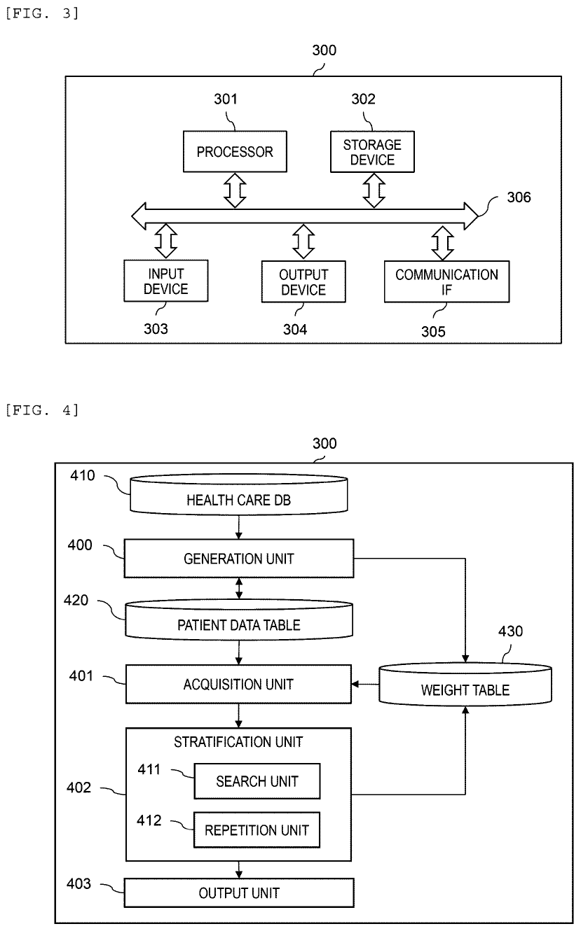

FIG. 3 is a block diagram showing a hardware configuration example of the analysis device. An analysis device 300 includes a processor 301, a storage device 302, an input device 303, an output device 304, and a communication interface (communication IF) 305. The processor 301, the storage device 302, the input device 303, the output device 304, and the communication IF 305 are connected to one another by a bus 306. The processor 301 controls the analysis device 300. The storage device 302 is a work area of the processor 301. In addition, the storage device 302 is a non-transitory or transitory recording medium that stores various programs or data. Examples of the storage device 302 include a read only memory (ROM), a random access memory (RAM), a hard disk drive (HDD), and a flash memory. The input device 303 inputs data. Examples of the input device 303 include a keyboard, a mouse, a touch panel, a numeric keypad, a scanner, a microphone, and a sensor. The output device 304 outputs data. Examples of the output device 304 include a display, a printer, and a speaker. The communication IF 305 is connected to a network to transmit and receive data.

### <Functional Configuration Example of Analysis Device>

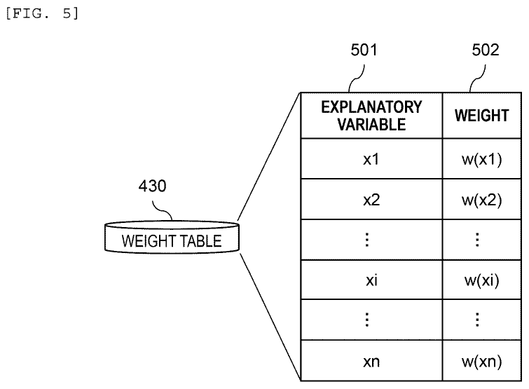

FIG. 4 is a block diagram showing a functional configuration example of the analysis device. The analysis device 300 includes a generation unit 400, an acquisition unit 401, a stratification unit 402, an output unit 403, a health care DB 410, a patient data table 420, and a weight table 430. Specifically, the health care DB 410, the patient data table 420, and the weight table 430 have, for example, a data structure stored in the storage device 302 shown in FIG. 3, and can be accessed by the processor 301. Specifically, for example, the generation unit 400, the acquisition unit 401, the stratification unit 402, and the output unit 403 are functions implemented by causing the processor 301 to execute a program stored in the storage device 302 shown in FIG. 3.

The generation unit 400 generates the patient data table 420 with reference to the health care DB 410. The acquisition unit 401 acquires a plurality of pieces of patient data specifying a patient from the patient data table 420 and acquires a weight from the weight table 430. The stratification unit 402 stratifies the patient group acquired as patient data by the acquisition unit 401. The stratification unit 402 includes a search unit 411 and a repetition unit 412. The search unit 411 searches for a branch condition for stratifying the patient group. The repetition unit 412 repeatedly executes the search for the branch condition by the search unit 411 and the division of the patient group using the branch condition. The output unit 403 outputs a stratification result obtained by the stratification unit 402.

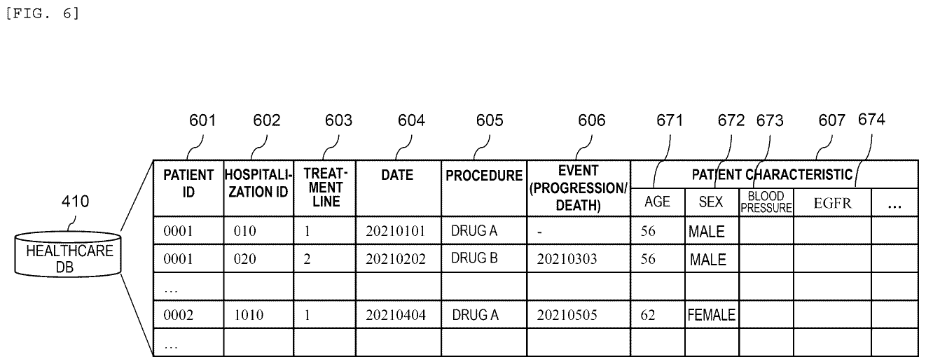

FIG. 5 is a diagram showing an example of the weight table 430 shown in FIG. 4. The weight table 430 includes an explanatory variable 501 and a weight 502 as fields. A combination of a value of the explanatory variable 501 and a value of the weight 502 in the same row is an entry for specifying one explanatory variable 501.

As described above, the explanatory variable 501 is a field for specifying a factor reflecting the sensitivity to the treatment, and holds x1, x2, . . . , xi, . . . , xn (n is an integer of 1 or more, and i is an integer satisfying 1≤i≤n) as identification information for uniquely specifying a predictive factor from among a certain number of explanatory variables. Hereinafter, the value of the explanatory variable 501 may be referred to as a predictive factor xi. The weight 502 is an index value indicating significance of the treatment effect τ, and is input to the above formula (1). In this embodiment, as the value of the weight 502 is larger, the prediction accuracy of the treatment effect τ is improved.

In Embodiment 1, the weight table 430 is prepared in advance. The analysis device 300 can execute addition, change, or deletion of an entry of the weight table 430 or change of the value of the weight 502 by an operation of a user.

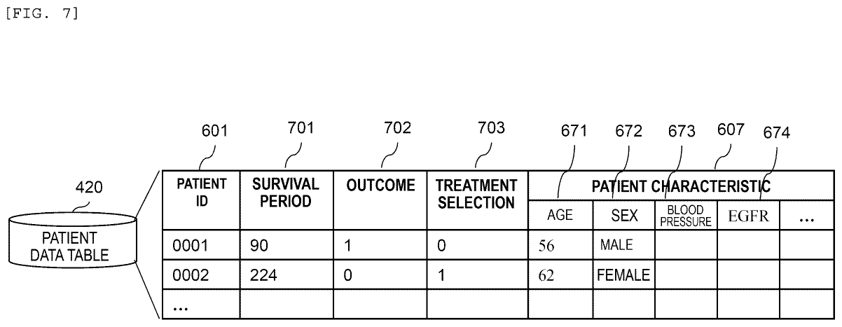

FIG. 6 is a diagram showing an example of the health care DB 410 shown in FIG. 4. The health care DB 410 includes, as fields, a patient ID 601, a hospitalization ID 602, a treatment line 603, a date 604, a procedure 605, an event 606, and a patient characteristic 607. A combination of values of the fields in the same row is an entry that defines one piece of health care information. There are one or more entries for one patient. For example, when a patient is hospitalized three times, three entries are present for the patient. In FIG. 6, health care information about injury or illness (for example, cancer) to be analyzed is defined.

The patient ID 601 is identification information for uniquely specifying a patient. The hospitalization ID 602 is identification information assigned when the patient specified by the patient ID 601 is hospitalized. The treatment line 603 is a number indicating an order of treatments.

The treatment line 603 is a number indicating an order of treatments for administration of an anticancer drug in a treatment for cancer. For example, when an anticancer drug is administered for a first time to a certain carcinoma, a value of the treatment line 603 is “1” for a first treatment, is “2” for a second treatment, and is “3” for a third treatment, and the like.

The date 604 is a year, a month, and a day when the treatment is performed by the treatment line 603. The procedure 605 is a content of the treatment of the treatment line 603. The event 606 is a result obtained by performing the procedure 605 in the treatment line 603 (for example, progression or death).

The patient characteristic 607 is an explanatory variable indicating a factor group serving as a feature of the patient specified by the patient ID 601 at a time point of the date 604, and includes a covariate. Specifically, the patient characteristic 607 is a clinical test value and the presence or absence of gene mutation, and includes, for example, an age 671, a sex 672, a blood pressure 673, and an EGFR 674 as factors.

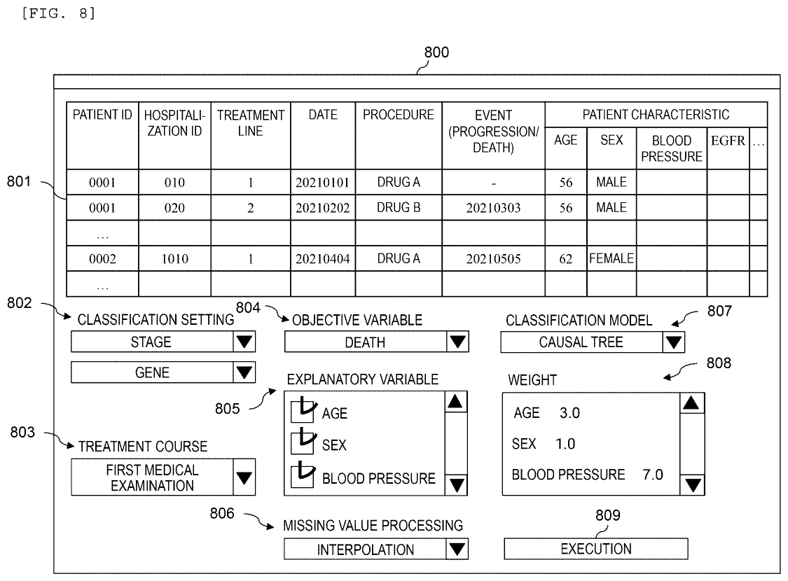

FIG. 7 is a diagram showing an example of the patient data table. The patient data table 420 is generated by the acquisition unit 401 with reference to the health care DB 410. The patient data table 420 may be stored in the storage device 302 in advance.

The patient data table 420 is a table in which the health care DB 410 is summarized in a patient unit, and includes, for example, the patient ID 601, a survival period 701, an outcome 702, a treatment selection 703, and the patient characteristic 607 as fields. A combination of values of the fields in the same row is an entry that defines patient data of one patient.

When there is a plurality of entries for one patient in the health care DB 410, for example, an entry in which the treatment line 603 has a maximum value is used as the entry of the patient data table 420.

The survival period 701 is the number of days of the patient specified by the patient ID 601 from the date 604 to a death date which is a value of the event 606. If there is no value in the event 606, the number of days is from the date 604 to the current date.

The outcome 702 is, for example, an observed value such as survival, progression-free survival, or a tumor size, and is a value inherently including a non-treatment-related effect and a treatment effect. Here, in the example of FIG. 7, the value of the outcome 702 is a numerical value for specifying survival. For example, “1” indicates survival and “0” indicates death. The analysis device 300 refers to the event 606, stores “1” when there is no value in the event 606, and stores “0” when there is a death date in the event 606.

The treatment selection 703 is a value indicating whether the patient specified by the patient ID 601 has selected a treatment, with “1” indicating that a treatment is selected and “0” indicating that a treatment is not selected. The analysis device 300 refers to the procedure 605, stores “0” when there is no value in the procedure 605, and stores “1” when there is a value in the procedure 605.

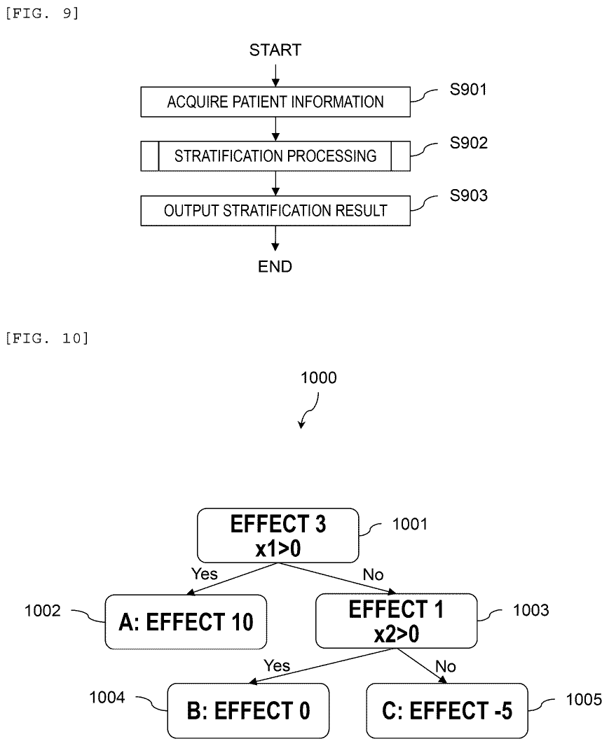

FIG. 8 is a diagram showing an example of an input screen of the analysis device 300. An input screen 800 is displayed on a display device, which is an example of the output device 304 of the analysis device 300, or on a display device of another computer that can communicate with the analysis device 300 via the communication IF 305. In addition, a user can input information to the input screen 800 by operating the input device 303 of the analysis device 300 or an input device of another computer.

The input screen 800 includes a health care information setting item 801, a classification setting item 802, a treatment course item 803, an objective variable item 804, an explanatory variable item 805, a missing value processing item 806, a classification model item 807, a weight item 808, and an execution button 809.

The health care information setting item 801 is a user interface capable of selecting a prediction target entry from an entry group of the health care DB 410 shown in FIG. 6. The classification setting item 802 is a user interface capable of selecting an item for classifying an entry group of the health care information setting item 801 by classification information such as a cancer stage or a gene of a patient. Accordingly, it is possible to narrow down the entry group of the health care information setting item 801. The treatment course item 803 is a user interface capable of selecting the treatment line 603 of the patient.

The objective variable item 804 is a user interface capable of selecting an objective variable output from a classification model f. As the objective variable, for example, the event 606 or the procedure 605 of the patient to be predicted can be selected. The explanatory variable item 805 is a user interface capable of selecting a factor of the patient characteristic 607 which is one or more explanatory variables of the patient to be predicted. In the example of FIG. 8, the age 671, the sex 672, and the blood pressure 673 are selected by inputting check marks.

The missing value processing item 806 is a user interface capable of selecting missing value processing of the explanatory variable. In the example of FIG. 8, “interpolation” is selected as the missing value processing. The classification model item 807 is a user interface capable of selecting the classification model f. In the example of FIG. 8, a causal tree is selected as the classification model f.

The weight item 808 displays the weight 502 of the explanatory variable corresponding to the explanatory variable 501 among the explanatory variables selected in the explanatory variable item 805. The user may not select an explanatory variable in the explanatory variable item 805 by referring to the weight 502. For example, since the weight 502 of the sex 672 is “1.0”, which is lower than the other weights 502, the user may exclude the sex 672 from the explanatory variable item 805. The execution button 809 is a user interface for causing the analysis device 300 to execute analysis processing by being pressed.

### <Analysis Processing>

FIG. 9 is a flowchart showing an analysis processing procedure example performed by the analysis device 300. The analysis device 300 generates the patient data table 420 based on the health care DB 410 when the patient data table 420 is not generated by the acquisition unit 401. Then, the analysis device 300 causes the acquisition unit 401 to acquire the patient data as the entry from the patient data table 420 (step S901).

Next, the analysis device 300 executes stratification processing by the stratification unit 402 (step S902). The stratification processing (step S902) is processing of stratifying patients using the patient data. Thereafter, the analysis device 300 outputs (step S903) a stratification result of the stratification processing (step S902) by the output unit 403, and ends the series of analysis processing. In step S903, the analysis device 300 may display the stratification result on a display which is an example of the output device 304, may transmit the stratification result to another computer through the communication IF 305, or may store the stratification result in the storage device 302.

### <Stratification Result>

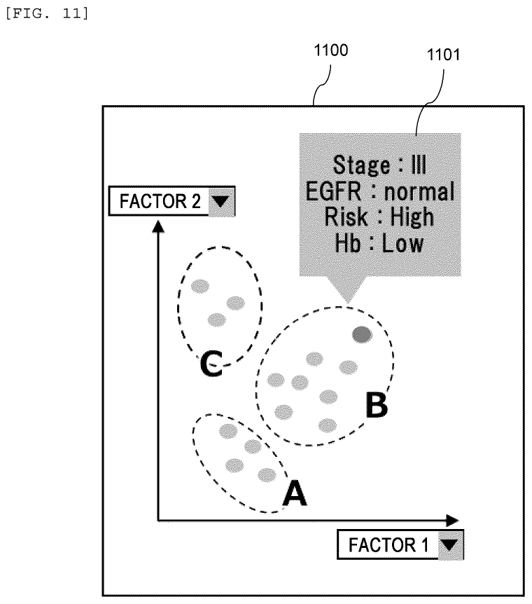

FIG. 10 is a diagram showing an example of the stratification result. The stratification result shown in FIG. 10 is a causal tree 1000 having a tree structure. The causal tree 1000 includes nodes 1001 to 1005. In the node 1001, an analysis target group in which an average value of treatment effects is “3” is divided into a patient group having a predictive factor x1>0 and a patient group having no predictive factor x1>0. The predictive factor x1 and a division threshold “0” for dividing the analysis target group are a branch condition of the node 1001. The patient group having the factor x1>0 is the node 1002 indicating the patient group A in which the average value of the treatment effects is “10”. The patient group having no factor x1>0 is the node 1003 in which the mean value of the treatment effects is “1”.

In the node 1003, a patient group as a division target, in which the mean value of the treatment effects is “1”, is divided into a patient group having a predictive factor x2>0 and a patient group having no predictive factor x2>0. The division threshold “0” for dividing the division target is a branch condition of the node 1003. The patient group having the predictive factor x2>0 is the node 1004 indicating the patient group B in which the mean value of the treatment effects is “0”. The patient group having no predictive factor x2>0 is the node 1005 indicating the patient group C in which the mean value of the treatment effects is “−5”.

There is no branch condition in the nodes 1002, 1004, and 1005. The nodes 1001 to 1005, a conjunction relationship between the nodes 1001 to 1005, and branch conditions of the nodes 1001 and 1003 constitute the causal tree 1000.

The division threshold is, for example, a value of a predictive factor for dividing the number of patients in the patient group as the division target into equal values. For example, the division threshold may be a minimum value of the predictive factor in the patient group in which the value of the predictive factor used for division is large, may be a maximum value of the predictive factor in the patient group in which the value of the predictive factor used for division is small, or may be a mean value of the minimum value of the predictive factor and the maximum value of the predictive factor.

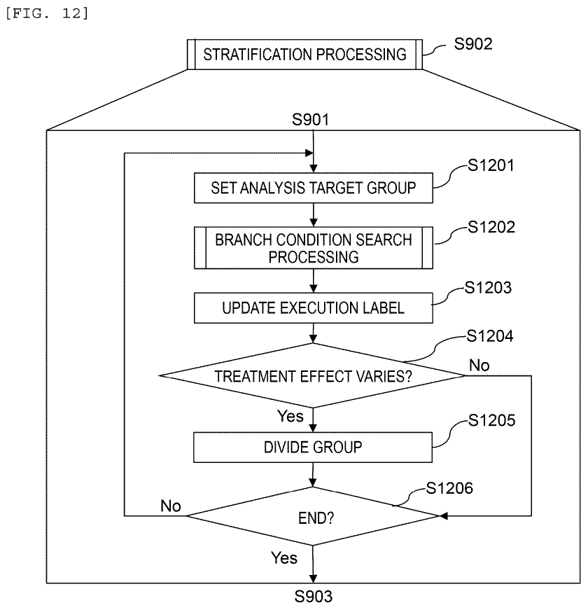

FIG. 11 is a diagram showing another example of the stratification result. A stratification result 1100 shown in FIG. 11 is an example shown in a graph. The stratification result 1100 is a scatter diagram that graphs a relationship between a factor 1 and a factor 2 which are the covariates. The analysis target group is divided into the patient groups A, B, and C. The covariate is not limited to a combination of the factor 1 and the factor 2, and other combinations can also be selected.

In addition, when the user operates the input device 303 to specify each of the patient groups A, B, and C, the analysis device 300 may display feature information of the specified patient group. In FIG. 11, when the patient group B is specified, feature information 1101 of the patient group B is displayed.

### <Stratification Processing>

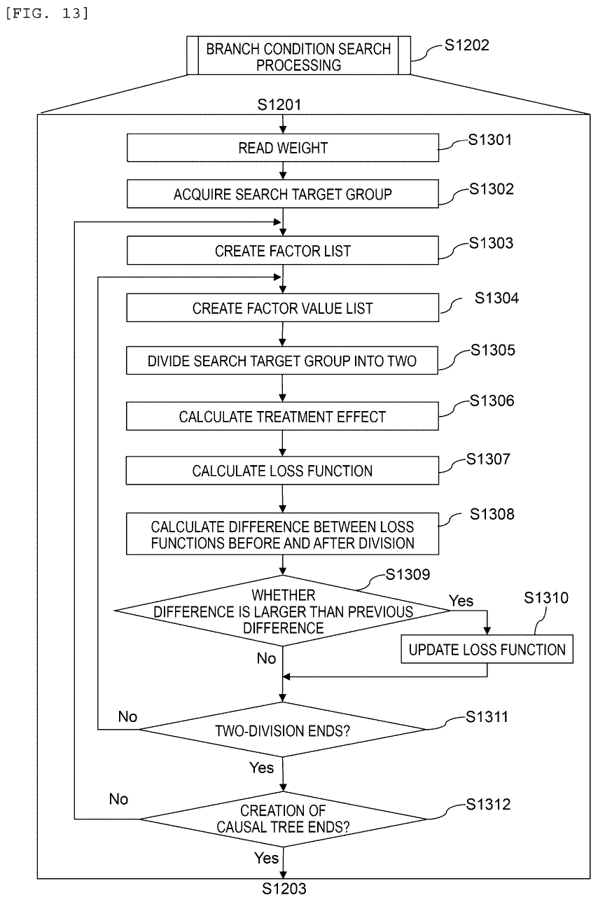

FIG. 12 is a flowchart showing a detailed processing procedure example of the stratification processing (step S902) shown in FIG. 9. The analysis device 300 sets an analysis target group by the repetition unit 412 (step S1201). Specifically, for example, during first execution of step S1201, the analysis device 300 selects the analysis target group at the time of the initial execution from the patient data acquired in step S901. The analysis target group during the first execution may be all entries of the patient data or the patient data table 420, may be a part of the patient data corresponding to a preset condition, or may be one or more pieces of patient data.

In addition, the analysis device 300 sets an execution label [K, V] in the analysis target group during the first execution of step S1201. For example, the execution label [K, V] is a combination of a key K and a value V. During the first execution of step S1201, the key K is set to 1 and the value V is set to False. False indicates that branch condition search processing (step S1202) is not executed, and when the branch condition search processing (step S1202) is executed, the value V is updated to Ture indicating that the branch condition search processing (step S1202) is executed.

Next, the analysis device 300 causes the search unit 411 to execute the branch condition search processing (step S1202). The branch condition search processing (step S1202) is processing of searching for a condition (branch condition) for branching the analysis target group and generating a causal tree.

Next, the analysis device 300 causes the search unit 411 to update the value V=False of the execution label [K, V] of the analysis target group to the value V=Ture indicating that the branch condition search processing (step S1202) is executed (step S1203).

Next, the analysis device 300 determines, by the repetition unit 412, whether the treatment effect varies before and after the division of the analysis target group (step S1204). Specifically, for example, the analysis device 300 temporarily divides the analysis target group, which is the division target, under a branch condition of the causal tree, and generates two patient groups (hereinafter, referred to as a first branch group and a second branch group, or simply referred to as branch groups when not distinguished). The analysis device 300 determines which of the first branch group and the second branch group has a treatment effect that significantly varies with respect to a treatment effect of the analysis target group which is the division target.

For example, the analysis device 300 calculates a standard deviation obtained by combining a treatment effect difference obtained by comparing the first branch group with the analysis target group (hereinafter referred to as a first difference) and a treatment effect difference obtained by comparing the second branch group with the analysis target group (hereinafter referred to as a second difference). Then, the analysis device 300 determines whether at least one of the first difference and the second difference is larger than the standard deviation.

It is determined that the treatment effect of the branch group as a comparison source whose difference is larger than the standard deviation varies from that of the analysis target group before division. Then, when at least one of the first difference and the second difference is larger than the standard deviation, it is determined that the treatment effect varies (step S1204: Yes), and the processing proceeds to step S1205. When both the first difference and the second difference are equal to or smaller than the standard deviation, the processing proceeds to step S1206.

In addition, in the branch condition search processing (step S1202), when the loss function is not improved (that is, when None is returned as a branch condition search result), the analysis device 300 determines that there is no variation in the treatment effect (step S1204: No) and proceeds to step S1206.

After step S1204: Yes, the analysis device 300 divides (step S1205) the analysis target group under the branch condition used in the temporary division in step S1204. Specifically, for example, the analysis device 300 divides the analysis target group at a parent node in the first step S1205, and when a loop is performed in step S1206: No, the analysis device 300 divides the analysis target group at a branch destination child node in the next step S1205.

In addition, the analysis device 300 assigns an execution label to each of the two groups divided in step S1205, that is, the first branch group and the second branch group. Specifically, for example, the analysis device 300 replicates the execution label [K, V] of the analysis target group for each of the first branch group and the second branch group. Then, the analysis device 300 assigns a branch number “1” to an end of the key K in the execution label [K, V] of the first branch group, and updates the value V from V=Ture to V=False. Similarly, the analysis device 300 assigns a branch number “2” to the end of the key K in the execution label [K, V] of the second branch group, and updates the value V from V=Ture to V=False.

For example, when the execution label [K, V] of the analysis target group is [1, Ture], the execution label [K, V] of the first branch group is [11, False], and the execution label [K, V] of the second branch group is [12, False]. Then, the processing proceeds to step S1206.

The analysis device 300 determines whether an end condition is satisfied (step S1206). The end condition is, for example, the number of times of execution of group division (step S1205) set in advance (that is, a depth of a branch) or a lower limit value of the number of samples in the group. Specifically, for example, when the number of times of execution of the group division (step S1205) is less than a predetermined number of times, it is determined that the end condition is not satisfied (step S1206: No), and the processing returns to step S1201. On the other hand, when the number of times of execution of the group division (step S1205) is equal to or more than the predetermined number of times, the value V of each of the first branch group and the second branch group is updated from V=False to V=Ture, it is determined that the end condition is satisfied (step S1206: Yes), the stratification processing (step S902) ends, and the processing proceeds to step S903.

In addition, when the end condition is the lower limit value of the number of samples in the group, the analysis device 300 determines whether the number of samples of each of the first branch group and the second branch group, which are obtained by the execution of the group division (step S1205), is less than the lower limit value of the number of samples in the group. When at least one of the first branch group and the second branch group is less than the lower limit value of the number of samples in the group, it is determined that the end condition is not satisfied (step S1206: No), and the processing returns to step S1201. On the other hand, when both the first branch group and the second branch group are equal to or more than the lower limit value of the number of samples in the group, the value V of each of the first branch group and the second branch group is updated from V=False to V=Ture, it is determined that the end condition is satisfied (step S1206: Yes), the stratification processing (step S902) ends, and the processing proceeds to step S903.

In addition, when the treatment effect does not vary (step S1204: No), the analysis device 300 determines whether the number of samples in the analysis target group is less than the lower limit value of the number of samples in the group. When the analysis target group is less than the lower limit value of the number of samples in the group, it is determined that the end condition is not satisfied (step S1206: No), and the processing returns to step S1201. On the other hand, when the analysis target group is equal to or more than the lower limit value of the number of samples in the group, the value V of each of the first branch group and the second branch group is updated from V=False to V=Ture, it is determined that the end condition is satisfied (step S1206: Yes), the stratification processing (step S902) ends, and the processing proceeds to step S903.

That is, when there is a group in which the value V of the execution label [K, V] is “False”, it is determined that the end condition is not satisfied (step S1206: No), and the processing returns to step S1201.

When the processing returns from step S1206: No to step S1201, the analysis device 300 sets the group, in which the value of the execution label [K, V] is “False”, as the next analysis target group (step S1201), and similarly executes steps S1202 to S1206.

In the example of the above group division (step S1205), the execution label [K, V] of the first branch group is [11, False], and the execution label [K, V] of the second branch group is [12, False]. Accordingly, the first branch group and the second branch group are respectively set as analysis target groups (step S1201), and steps S1202 to S1206 are executed for each analysis target group.

Here, the causal tree 1000 shown in FIG. 10 will be specifically described as an example. First, during the first execution, the analysis device 300 temporarily divides the analysis target group into the first branch group (x1>0: Yes) and the second branch group (x1>0: No) under the branch condition (x1>0) of the node 1001. Here, it is assumed that the treatment effect varies for either the first branch group (x1>0: Yes) or the second branch group (x1>0: No) (step S1204: Yes). Accordingly, the analysis device 300 divides the analysis target group into the first branch group (x1>0: Yes) and the second branch group (x1>0: No) under the branch condition (x1>0) of the node 1001 (step S1205).

In addition, the analysis device 300 generates the execution label [11, False] of the first branch group (x1>0: Yes) and the execution label [12, False] of the second branch group (x1>0: No) using the execution label [1, True] of the analysis target group.

The first branch group (x1>0: Yes) transitions to the node 1002. Since there is no branch condition in the node 1002, the analysis device 300 ends the search for the first branch group (x1>0: Yes) (step S1206: Yes), and updates the execution label [11, False] to an execution label [11, True].

The execution label of the second branch group (x1>0: No) is [12, False], and the value V is False. Accordingly, the analysis device 300 sets the second branch group (x1>0: No) as the next analysis target group (step S1206: No→S1201).

The analysis device 300 specifies the node 1002 to which the analysis target group (x1>0: No) transitions in the causal tree 1000, and updates the execution label [12, False] thereof to an execution label [12, True].

Then, the analysis device 300 temporarily divides the analysis target group (x1>0: No) into a third branch group (x2>0: Yes) and a fourth branch group (x2>0: No) under the branch condition (x2>0). Here, it is assumed that the treatment effect varies for either the third branch group (x2>0: Yes) or the fourth branch group (x2>0: No) (step S1204: Yes). The analysis device 300 divides the analysis target group (x1>0: No) into the third branch group (x2>0: Yes) and the fourth branch group (x2>0: No) under the branch condition (x2>0) (step S1205).

In addition, the analysis device 300 generates an execution label [123, False] of the third branch group (x2>0: Yes) and an execution label [124, False] of the fourth branch group (x2>0: No) using the execution label [12, True] of the analysis target group (x1>0: No).

The third branch group (x2>0: Yes) transitions to the node 1004. Since there is no branch condition in the node 1004, the analysis device 300 ends the search for the third branch group (x2>0: Yes) (step S1206: Yes), and updates the execution label [123, False] to an execution label [123, True].

Similarly, the fourth branch group (x2>0: No) transitions to the node 1005. Since there is no branch condition in the node 1005, the analysis device 300 ends the search for the fourth branch group (x2>0: No) (step S1206: Yes), and updates the execution label [124, False] to an execution label [124, True].

Then, the analysis device 300 outputs, as the stratification result, the execution labels generated so far, a group corresponding to the execution label, and a branch condition used for the division.

In step S903 of FIG. 9, the analysis device 300 causes the output unit 403 to output, for example, a causal tree having a tree structure from an initial analysis target group to a branch group at the end as the stratification result. At this time, the execution label of each group of the stratification result may be reassigned to an ascending order number starting from 0 using the initial analysis target group as a start position.

Accordingly, in the stratification processing (step S902), a search is executed to maximize the treatment effect for each branch group generated by branching, thereby implementing stratification in which the treatment effect is maximized.

### <Branch Condition Search Processing (Step S1002)>

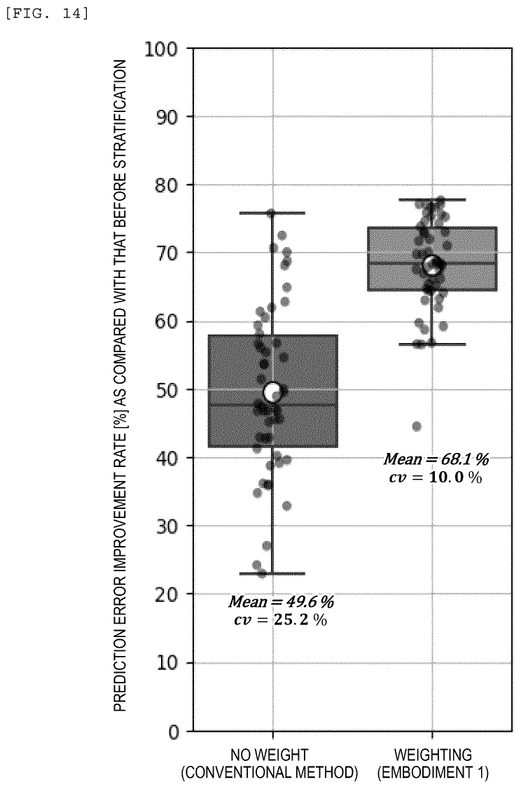

FIG. 13 is a flowchart showing a detailed processing procedure example of the branch condition search processing (step S1002) shown in FIG. 10. The search unit 411 reads the weight 502 of the explanatory variable 501 from the weight table 430 (step S1301).

Next, the search unit 411 acquires a search target group from the analysis target group (step S1302). Specifically, for example, the search unit 411 may set the analysis target group as the search target group as it is, or may divide the analysis target group into training data and verification data. In the case of the division, the training data becomes the search target group, and the verification data is used in treatment effect estimation (step S1306).

Next, the search unit 411 randomly selects factors that are covariates in the search target group, creates a list of the selected factors (factor list) (step S1303), and creates a list of values of the selected factors (factor value list) (step S1304). The factor list is a list of fields indicating factors that are covariates such as the age 671, the blood pressure 673, and the EGFR 674. The factor group selected in the factor list is a factor group in which the number is smaller than all the factors. A causal tree is created for each factor list.

The factor value list is a list including values (56 [years old], 62 [years old], . . . 90 [ml], 127 [ml], . . . ) of the selected factors such as the age 671, the blood pressure 673, and the EGFR 674.

In addition, in step S1304, the search unit 411 specifies a preset predictive factor from the factor list, and extracts a value of the specified predictive factor (hereinafter, search target predictive factor) from the factor value list.

Through steps S1301, S1303, and S1304, the search unit 411 selects unselected predictive factors and weights thereof.

Next, the search unit 411 divides the search target group into two by using the search target predictive factor (step S1305). The data division is processing of dividing the search target group into the subtypes L and R according to the patient characteristics shown in FIG. 2. Every time the processing returns from steps S1311 and S1312, a different predictive factor is selected as the search target predictive factor. Similar to FIG. 2, one of the divided groups is referred to as the subtype L, and the other group is referred to as the subtype R.

Next, the search unit 411 calculates the treatment effect τ for each of the subtypes L and R (step S1306). The treatment effect τ is calculated by the following formula (2).

$$τ ( 1 ) = E [ Y \vert T = 1 ] - E [ Y \vert T = 0 ] \qquad ( 2 )$$

For the subtype L, l=L, and for the subtype R, l=R. Y is an outcome (for example, the event 606). T is a binary variable indicating the treatment selection, T=1 indicates that the treatment is selected (the procedure 605 is performed), and T=0 indicates that the treatment is not selected (the procedure 605 is not performed). In addition, E[ ] is an expectation operator. E[ ] is, for example, a sum of an outcome Y. The treatment effects τ(L) and τ(R), which are second treatment effects, are calculated based on the above formula (2). When the treatment effects τ(L) and τ(R) are not distinguished, they are referred to as τ(l) (where l=L, R).

Next, the search unit 411 calculates a loss function before and after the division using the treatment effects τ(L) and τ(R) (step S1307). The loss function before the division is defined as Loss Pre, and the loss function after the division is defined as Loss Post. First, the loss function before the division Loss Pre is expressed by the following formula (3).

$$[ Math 2 ]$$

$$LossPre = N \cdot τ \times W ( x ) \qquad ( 3 )$$

In the above formula (3), N on the right side is the number of samples in the search target group. In addition, τ on the right side is a treatment effect before the division that is a first treatment effect. During the first execution, the treatment effect τ in a parent node is used. After the second loop, the treatment effect τ(l) after the previous division is the treatment effect τ before the division.

In addition, X is the search target predictive factor specified in step S1305 among the explanatory variables 501 (x1, x2, . . . , xi, . . . , xn). W(x) is the weight 502 of the search target predictive factor.

In addition, in step S1302, when the analysis target group is divided into the training data and the verification data, a penalty term based on a variance is added to the above formula (3), and the loss function before the division Loss Pre is the following formula (4).

$$[ Math 3 ]$$

$$LossPre = N \cdot τ \times W ( x ) - ( 1 + \frac{{N}_{train}}{{N}_{est}} ) * ( \frac{{S}_{T = 1}^{2}}{p} + \frac{{S}_{T = 0}^{2}}{1 - p} ) \qquad ( 4 )$$

Nₜᵣₐᵢₙ on the right side of formula (4) is the number of samples in the training data, that is, the number of samples N in the search target group. Nₑₛₜ is the number of samples in the verification data. S_{T=1} is a variance of samples belonging to a treatment selection T=1 in the search target group. S_{T=0} is a variance of samples belonging to a treatment selection T=0 in the search target group. In addition, p is a ratio of the number of samples belonging to the treatment selection T=1 in the search target group.

In addition, the entire right side of each of the formulas (3) and (4) may be divided by the number of samples N in the search target group and thus be normalized.

Next, the loss function after the division Loss Post is expressed by the following formula (5). The loss function after the division Loss Post is a loss function that maximizes the estimated treatment effect τ(l).

$$[ Math 4 ]$$

$$LossPost = {\sum }_{l}^{L , R} {N}_{l} \cdot {τ}_{l} \times W ( x ) \qquad ( 5 )$$

In formula (5), N(l) on the right side is the number of samples in a subtype l. When the entire right sides of the above formulas (3) and (4) are normalized by being divided by the number of samples N in the search target group, the entire right side of the above formula (5) may be normalized by being divided by the number of samples in the search target group (the total number of samples in the subtypes L and R). In addition, val is a threshold for partitioning the range of the factor x. W(x) may be used without using val.

Next, the search unit 411 calculates a difference Gain between the loss function before the division Loss Pre and the loss function after the division Loss Post (step S1308). The difference Gain is an index indicating whether the loss function Loss Post is improved by division.

$$Gain = Loss Post - Loss Pre \qquad ( 6 )$$

Next, the search unit 411 determines whether the current difference Gain is larger than a retained difference Gain (step S1309). The retained difference Gain is a difference Gain retained in step S1310 in the previous loop, and is a target value. However, since there is no retained difference Gain during the first execution, 0 is used as an initial value of the retained difference Gain.

If the current difference Gain is larger than the retained difference Gain (step S1309: Yes), the search unit 411 updates the currently applied loss function before the division Loss Pre with the loss function Loss Post, sets the currently applied loss function before the division Loss Pre as a new loss function before the division Loss Pre, updates the retained difference Gain with the current difference Gain, and acquires the branch condition when the division into two at step S1305 is executed. Accordingly, the branch condition is searched. Then, the processing proceeds to step S1311.

On the other hand, when the current difference Gain is not larger than the retained difference Gain (step S1309: No), the search unit 411 proceeds to step S1311 without updating the loss function before the division Loss Pre and updating the retained difference Gain.

Next, the search unit 411 determines whether the division of the search target group into two (step S1305) satisfies the end condition (step S1311). The end condition is, for example, a case where there is no explanatory variable 501 that can be selected as a search target. When the division of the search target group into two (step S1305) does not satisfy the end condition (step S1305: No), that is, when the explanatory variable 501 that can be selected as the search target remains, the processing returns to step S1304. In this case, the search unit 411 sets, as the next search target group, each of the subtypes L and R determined to be larger than the previous difference in step S1309.

On the other hand, when the end condition is satisfied (step S1311: Yes), that is, when the explanatory variable 501 that can be selected as the search target does not remain, one causal tree is created, the search unit 411 stores the created causal tree, and the processing proceeds to step S1312.

Next, the search unit 411 determines whether the end condition for the creation of the causal tree is satisfied (step S1312). The end condition is, for example, a threshold of the number of causal trees. When the end condition is not satisfied (step S1312: No) (when the number of created causal trees does not reach the threshold), the processing returns to step S1303, and the search unit 411 recreates a factor list.

On the other hand, when the end condition is satisfied (step S1312: Yes), the search unit 411 outputs the created causal tree, and the processing proceeds to step S1203. Accordingly, a causal tree corresponding to the threshold set in step S1312 is created. A node having a branch destination node among a node group constituting the causal tree includes a predictive factor and a division threshold used when the node group is divided into groups by the node.

### <Simulation Result>

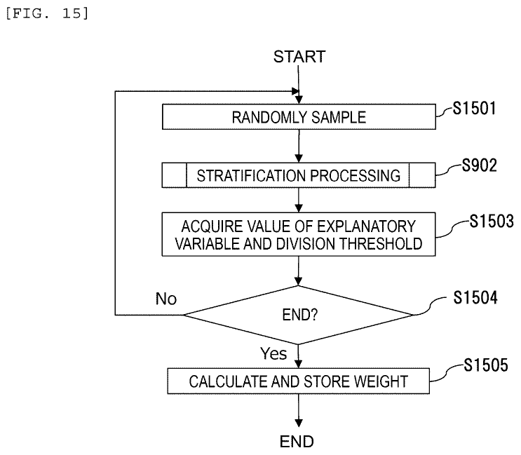

Next, a simulation result of Embodiment 1 will be described with reference to FIG. 14.

FIG. 14 is a box plot diagram showing a prediction error improvement rate as compared with a case before division between a conventional method and Embodiment 1. The conventional method is a method of calculating the prediction error improvement rate by a formula obtained by excluding W(x) from the formulas (3) and (5).

$${Y}_{j} = η ( {x}_{j} ) + {T}_{j} \cdot τ ( {x}_{j} ) \qquad ( 7 )$$

The above formula (7) is an outcome calculation formula. An additional character j is the patient ID 601. Yⱼ on the left side is an outcome of a patient for which a value of the patient ID 601 is j (hereinafter, patient j). η(xⱼ) is a non-treatment-related effect by a prognostic factor xⱼ of the patient j. Tⱼ is the treatment selection T(=0 or 1) of the patient j. τ(xⱼ) is a treatment effect by the predictive factor xⱼ.

Here, η(x) is expressed by the following formula (8).

$$[ Math 5 ]$$

$$η ( {x}_{j} ) = 0.5 ( {\sum }_{j = 1}^{j = 4} {x}_{j} ) + {\sum }_{j = 5}^{j = 8} {x}_{j} \qquad ( 8 )$$

In addition, τ(xⱼ) is expressed by the following formula (9).

$$[ Math 6 ]$$

$$τ ( {x}_{j} ) = 3 ( {\sum }_{j = 1}^{j = 2} {x}_{j} ) \qquad ( 9 )$$

The above formulas (8) and (9) are formulas indicating a data generation method performed by simulation, and table data similar to FIG. 7 is created. The number of samples N of the patient j is set to N=1000, and a treatment selection Tⱼ of the patient j is random. Here, it is assumed that among the factors x1 to x8, the factors x1 and x2 have a value of the weight 502 that is significantly larger than those of the other factors x3 to x8.

In this simulation, a prediction error reduction rate before and after the division is calculated using a root mean square error (RMSE) as an evaluation of accuracy. In Embodiment 1, since weighting is performed, it can be confirmed that the prediction error improvement rate is improved and a coefficient of variation (CV) is remarkably reduced.

### Embodiment 2

Next, Embodiment 2 will be described. Embodiment 1 has been described on the assumption that the weight table 430 is present, but Embodiment 2 is an example in which the analysis device 300 generates the weight table 430. That is, in Embodiment 2, the analysis device 300 causes the generation unit 400 to generate the weight table 430 by referring to the patient data table 420. In Embodiment 2, differences from Embodiment 1 will be mainly described, and thus description of the same parts as those in Embodiment 1 will be omitted.

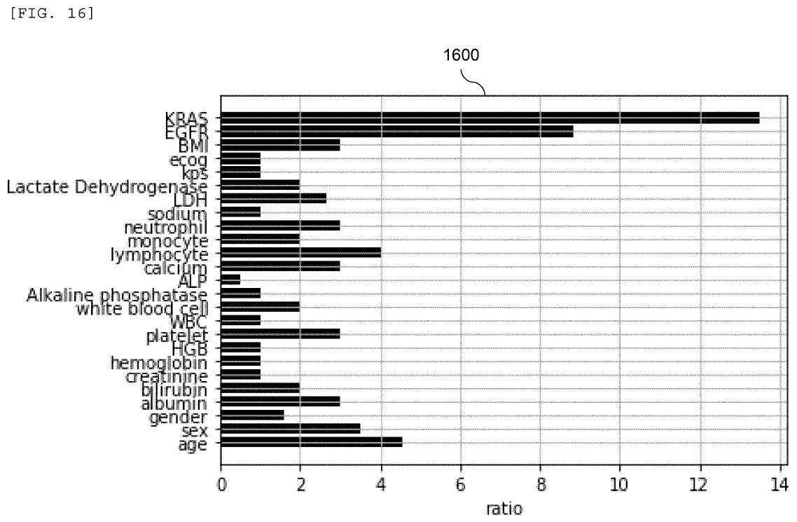

FIG. 15 is a flowchart showing a generation processing procedure example of the weight table 430 generated by the generation unit 400 according to Embodiment 2. The generation unit 400 randomly samples an entry that defines patient data based on the patient data table 420 (step S1501). The sampling number is freely set to, for example, 50% or 70% of all samples in the patient data table 420. In addition, the generation unit 400 may use, as the verification data, a sample that has not been sampled.

Next, the generation unit 400 outputs a sample group sampled at step S1501 to the stratification unit 402, and calls and executes the stratification processing (step S902) shown in FIG. 12 from the stratification unit 402 (step S902).

Next, the generation unit 400 acquires, from each branch group that is the stratification result of the stratification processing (step S902), the value of the explanatory variable 501 and the division threshold thereof for each explanatory variable 501 used for division (step S1503).

Thereafter, the generation unit 400 determines whether the end condition is satisfied (step S1504). Specifically, the end condition is, for example, a case where the number of times of execution of steps S1501 to S1503 reaches a predetermined number of times. When the end condition is not satisfied (step S1504: No), that is, when the number of times of execution of steps S1501 to S1503 has not reached the predetermined number of times, the processing returns to step S1501. On the other hand, when the end condition is satisfied (step S1504: Yes), that is, when the number of times of execution of steps S1501 to S1503 reaches the predetermined number of times, the weight 502 is calculated for each explanatory variable 501 and stored in the weight table 430 (step S1505).

Specifically, for example, the generation unit 400 calculates, for each explanatory variable 501, a statistic between the value of the explanatory variable 501 and the division threshold, and sets the calculated value as the weight 502. More specifically, for example, a difference between a maximum value of the values of the explanatory variable 501 and the division threshold may be set as the weight 502, a difference between a median value of the values of the explanatory variable 501 and the division threshold may be set as the weight 502, a difference between a mode value of the values of the explanatory variable 501 and the division threshold may be set as the weight 502, and a difference between a mean value of the values of the explanatory variable 501 and the division threshold may be set as the weight 502. In addition, the number of appearances of the value of the explanatory variable 501 may be used.

Accordingly, the analysis device 300 automatically learns the weight as medical knowledge. Accordingly, the weight 502 can be increased for the predictive factor used as the branch condition, and the estimation accuracy of the treatment effect can be improved.

Since the above stratification processing (step S902) is also applied to FIG. 9, when the stratification processing (step S902) is executed in FIG. 9, the generation unit 400 may update the weight table 430 using the stratification result. Accordingly, as the analysis is performed by the analysis device 300, the reliability of the weight table 430 is improved, and the estimation accuracy of the treatment effect is improved.

In addition, in Embodiment 1, the freely created weight table 430 is applied, but in Embodiment 2, a computer including the generation unit 400 other than the analysis device 300 may generate the weight table 430 by the generation processing according to Embodiment 2, and the analysis device 300 may acquire the weight table 430 from the computer.

### Embodiment 3

Next, Embodiment 3 will be described. Embodiment 1 has been described on the assumption that the weight table 430 is present, but Embodiment 3 is an example in which the analysis device 300 generates the weight table 430. That is, in Embodiment 3, the analysis device 300 causes the generation unit 400 to generate the weight table 430 by referring to a medical literature database such as PubMed. In Embodiment 3, differences from Embodiment 1 will be mainly described, and thus description of the same parts as those in Embodiment 1 will be omitted.

Specifically, for example, the analysis device 300 causes the generation unit 400 to execute abstract search on the medical literature database, statistically process appearance rates of related phrases, and set a statistical processing result to the weight 502 of the explanatory variable 501. Accordingly, the analysis device 300 automatically learns medical knowledge.

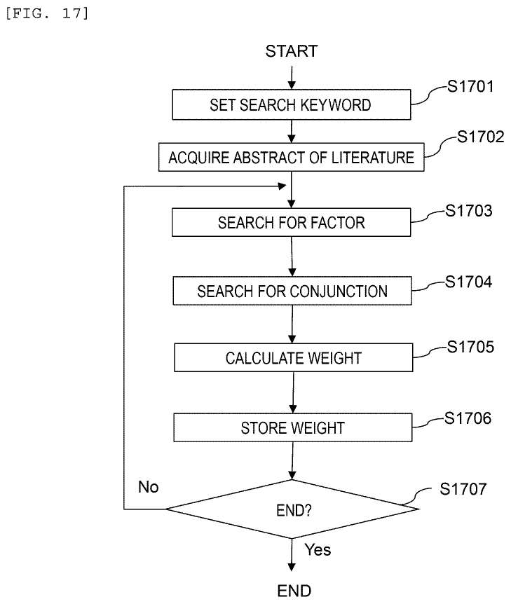

FIG. 16 is a histogram showing a search result from the medical literature database. The vertical axis in a histogram 1600 is a string of factors included in a sentence searched for by a search keyword. For example, a name of a risk factor is used as the search keyword. In addition, the search keyword may include a conjunction related to an outcome such as “cause” or “relate”.

The horizontal axis in FIG. 16 is the weight 502 of the factor. The generation unit 400 calculates the value of the weight 502 such that the value increases as the number of appearances of the search keyword in the sentence searched for by the search keyword or the number of sentences searched for by the search keyword increases. However, when a negative word such as “not” is included in the sentence searched for by the search keyword, the generation unit 400 calculates the value of the weight 502 not to be high or low.

The generation unit 400 excludes the factors of which the value of the weight 502 is equal to or less than a predetermined threshold or the upper (k+1)-th and succeeding factors, and stores, as the explanatory variables 501, the factors of which the value of the weight 502 is larger than the predetermined threshold or the factors up to the k-th factor into the weight table 430 together with the weight 502.

FIG. 17 is a flowchart showing a generation processing procedure example of the weight table 430 according to Embodiment 3. The generation unit 400 sets a search keyword by a user operation (step S1701). Next, the generation unit 400 transmits the search keyword to the medical literature database, searches for an abstract of each literature in the medical literature database, and acquires an abstract of a literature corresponding to the search keyword from the medical literature database (step S1702).

Next, the generation unit 400 searches for the abstract acquired in step S1702 by the factor included in the search keyword, and extracts a sentence including the factor (step S1703).

Next, the generation unit 400 searches for the sentence extracted in step S1703 by a conjunction (for example, “cause” or “relate”) related to the outcome, and increments a positive relationship count Cpos for the sentence including the conjunction. The positive relationship count Cpos is an evaluation value related to a sentence indicating that a relation between a factor and a conjunction is positive, and the weight 502 increases as a count value increases. On the other hand, when a negative word such as “not” is included in the sentence searched for by the conjunction related to the outcome, the generation unit 400 increments a negative relationship count Cneg.

Next, the generation unit 400 calculates the weight 502 for each factor (step S1705). The weight 502 (w) is calculated by, for example, the following formula (10).

$$w = Cpos / Cneg \qquad ( 10 )$$

When the negative relationship count Cneg of the denominator is not counted even once, Cneg=0 and the calculation becomes impossible, and thus the formula (1) may be corrected such that the denominator of the formula (10) does not become 0 even when Cneg=0.

Next, the generation unit 400 stores the calculated weight 502 in the weight table 430 (step S1706).

Thereafter, the generation unit 400 determines whether an end condition is satisfied (step S1704). Specifically, the end condition is, for example, a case where all the weights 502 have been calculated for the factors searched for in step S1703. When there is a factor for which the weight 502 is not calculated (step S1707: No), the processing returns to step S1703. On the other hand, when there is no factor for which the weight 502 is not calculated (step S1707: Yes), the generation unit 400 ends an example of processing.

Accordingly, the analysis device 300 automatically learns the medical knowledge as a weight. Accordingly, the weight 502 is larger for a factor searched from the medical literature database, and when the factor and the predictive factor have medical basis from the medical literature, the estimation accuracy of the treatment effect can be improved.

In Embodiment 3, since the abstract of the medical literature is used as a search target, it is possible to speed up the generation processing of the weight table 430 as compared with the case of using the medical literature as the search target. On the other hand, the generation unit 400 may use the medical literature as the search target. Accordingly, the reliability of the weight 502 is improved and the estimation accuracy of the treatment effect is improved as compared with the case where the abstract of the medical literature is used as the search target.

In addition, in Embodiment 1, the freely created weight table 430 is applied, but in Embodiment 1, a computer including the generation unit 400 other than the analysis device 300 may generate the weight table 430 by the generation processing according to Embodiment 3, and the analysis device 300 may acquire the weight table 430 from the computer.

As described above, according to the analysis device 300 described above, classification accuracy in the case where patients are stratified by factors contributing to the treatment effect is improved by weighting predictive factors estimated from experience or medical literatures in advance. Accordingly, the estimation accuracy of the treatment effect is improved, and more correct patient stratification can be implemented.

Accordingly, the analysis device 300 can directly classify the patients into subtypes based on the estimated treatment effect corresponding to the patient characteristic. Accordingly, the stratified patient groups are classified as subtypes having different treatment effects, and are expected to contribute to an optimal treatment selection tailored an to individual characteristic of a patient. Accordingly, it is possible to specify a subtype that can be expected to have a treatment effect from a certain drug.

The invention is not limited to the above embodiments, and includes various modifications and equivalent configurations within the scope of the appended claims. For example, the above embodiments are described in detail for easy understanding of the invention, and the invention is not necessarily limited to those including all the configurations described above. A part of a configuration of one embodiment may be replaced with a configuration of another embodiment. A configuration of one embodiment may also be added to a configuration of another embodiment. Another configuration may be added to a part of a configuration of each embodiment, and a part of the configuration of each embodiment may be deleted or replaced with another configuration.

A part or all of the above configurations, functions, processing units, processing methods, and the like may be implemented by hardware by, for example, designing with an integrated circuit, or may be implemented by software by, for example, a processor interpreting and executing a program for implementing each function.

Information on such as a program, a table, and a file for implementing each function can be stored in a storage device such as a memory, a hard disk, or a solid state drive (SSD), or in a recording medium such as an integrated circuit (IC) card, an SD card, or a digital versatile disc (DVD).

Control lines and information lines considered to be necessary for description are shown, and not all control lines and information lines necessary for implementation are shown. Actually, almost all components may be considered to be connected to one another.

1. An analysis device comprising:
a processor configured to execute a program; and
a storage device configured to store the program, wherein
the storage device stores a weight for each predictive factor group in a factor group, and
the analysis device includes
an acquisition unit configured to acquire a plurality of pieces of patient data including a value for each factor of the factor group for each patient, and
a search unit configured to repeatedly execute selection processing of selecting the factor and the weight, division processing of dividing the plurality of pieces of patient data that are a division target based on the factor and the weight that are selected by the selection processing, and setting processing of setting a patient data group obtained by the division processing as a new division target, thereby executing search processing of searching for a branch condition for dividing the division target by the division processing.

2. The analysis device according to claim 1, wherein
the patient data includes a variable related to a treatment selection indicating whether the patient selects a treatment, and
the search unit executes, when the plurality of pieces of patient data are set as the division target by the setting processing, treatment effect calculation processing of calculating a first treatment effect related to the factor using the variable for the plurality of pieces of patient data and calculating a second treatment effect related to the factor using the variable for each of two patient data groups divided by the division processing, loss function calculation processing of calculating a loss function before division based on the first treatment effect, the factor, and the weight and calculating a loss function after division based on the second treatment effect, the factor, and the weight for each of the two patient data groups, and difference calculation processing of calculating a difference between the loss function before division and the loss function after division, and searches for the branch condition based on the difference.

3. The analysis device according to claim 2, wherein
when the difference is larger than a target value, the search unit executes update processing of updating the loss function before division with the loss function after division and updating the target value with the difference.

4. The analysis device according to claim 2, wherein
the search unit executes the search processing using the plurality of pieces of patient data as an analysis target group, and
the analysis device includes a stratification unit configured to execute stratification processing of temporarily dividing the analysis target group into a first branch group and a second branch group under the branch condition based on the predictive factor and the weight, and dividing, by executing determination processing of determining whether the second treatment effect of any of the first branch group and the second branch group significantly varies based on a comparison result between the first treatment effect of the analysis target group and the second treatment effect for the first branch group and a comparison result between the first treatment effect of the analysis target group and the second treatment effect for the second branch group, the analysis target group into the first branch group and the second branch group based on a determination result of the determination processing.

5. The analysis device according to claim 4, wherein
the stratification unit executes the stratification processing using at least one or more pieces of patient data among the plurality of pieces of patient data as the analysis target group, and
the analysis device includes a generation unit configured to generate the weight of the factor based on a branch condition when the one or more pieces of patient data are divided into the first branch group and the second branch group by the stratification processing.

6. The analysis device according to claim 1, further comprising:
a generation unit configured to search a medical literature database with a search keyword including the factor and a conjunction related to an outcome, calculate a weight of the factor included in the search keyword by extracting a sentence corresponding to the search keyword, and store the factor included in the search keyword in the storage device in association with the weight.

7. The analysis device according to claim 1, wherein
the factor is a predictive factor that reflects sensitivity to treatment.

8. An analysis method executed by an analysis device including a processor that executes a program and a storage device that stores the program, wherein
the storage device stores a weight for each predictive factor group in a factor group, and
the processor executes
acquisition processing of acquiring a plurality of pieces of patient data including a value for each factor of the factor group for each patient, and
search processing of repeatedly executing selection processing of selecting the factor and the weight, division processing of dividing the plurality of pieces of patient data that are a division target based on the factor and the weight that are selected by the selection processing, and setting processing of setting a patient data group obtained by the division processing as a new division target, thereby executing search processing of searching for a branch condition for dividing the division target by the division processing.

9. An analysis program that causes a processor, which is accessible to a storage device storing a weight for each predictive factor of a factor group among factor groups, to execute
acquisition processing of acquiring a plurality of pieces of patient data including a value for each factor of the factor group for each patient, and
search processing of repeatedly executing selection processing of selecting the factor and the weight, division processing of dividing the plurality of pieces of patient data that are a division target based on the factor and the weight that are selected by the selection processing, and setting processing of setting a patient data group obtained by the division processing as a new division target, thereby executing search processing of searching for a branch condition for dividing the division target by the division processing.
